(19) United States
(12) Patent Application Publication
(10) Pub. No.: US 2017/0143548 A1
(43) Pub. Date: May 25, 2017

(54) WELDING HELMET HAVING AUTOMATIC WIRELESS LIGHT-BLOCKING MEANS AND CONTROL METHOD THEREFOR

(71) Applicant: Dong Hun LEE, Seongnam-si (KR)

(72) Inventor: Dong Hun LEE, Seongnam-si (KR)

(21) Appl. No.: 15/309,924

(22) PCT Filed: Jan. 12, 2015
(86) PCT No.: PCT/KR2015/000296

§ 371 Date: Nov. 9, 2016

(30) Foreign Application Priority Data
May 13, 2014 (KR) 10-2014-0057316

(51) Int. Cl. A61F 9/06; A42B 3/30; A42B 3/24

(52) U.S. Cl. CPC A61F 9/061; A61F 9/065; A42B 3/24; A42B 3/30

(57) ABSTRACT

The present invention relates to a welding helmet including a wireless auto light shield part and a controlling method thereof. More particularly, the present invention relates to a welding helmet including a wireless auto light shield part capable of improving convenience of a welding operation, protecting eyes of a worker from direct light during welding operation, and preventing frost from being formed on the viewing window by shielding or opening a viewing window through elevation of a shield glass while automatically driving the light shield part in a wireless scheme by operation of a torch switch, and a method of controlling the same.

### BACKGROUND OF THE INVENTION

Field of the Invention

The present invention relates to a welding helmet including a wireless auto light shield part and a controlling method thereof. More particularly, the present invention relates to a welding helmet including a wireless auto light shield part capable of improving convenience of a welding operation, protecting eyes of a worker from direct light during welding operation, and preventing frost from being formed on the viewing window by shielding or opening a viewing window through elevation of a shield glass while automatically driving the light shield part in a wireless scheme by operation of a torch switch, and a method of controlling the same.

Related Art

In general, welding is a process which directly combines the same type of different types of metallic materials with each other by locally applying heat or pressure to the metallic materials. Since strong light is emitted and fragments are bounced upon a welding operation, if the worker's eyes are exposed to light, a sight may be damaged and the worker' eyeballs may be damaged due to fragments.

Accordingly, when the welding operation is performed, strong light is shielded for protecting a welder's eyes from harmful materials, and the welder performs a welding operation while wearing a welding helmet to protect the worker' face from spark to be generated during the welding.

A welding helmet according to the related art includes a face protection surface configured to protect a face of a worker from spark fragments scattered during a welding operation, a viewing window located in the face protection surface so that the worker may perform the welding operation while directly confirming a welding region, and a wearing unit configured to allow a worker to perform the welding operation while adhering the face protection surface to the worker' face. The viewing window is provided therein with a light shield glass.

After the worker recognizes a welding position and a relation position between an object to be welded and a welding rod by opening the viewing window after starting the welding, the worker' eyes are protected from direct light emitted during welding by shielding the viewing window by the light shield glass after starting the welding when the welding starts.

According to the related art, the sight is ensured by lifting the shield glass upward from the face protection surface before the welding starts. During the welding, an operation of shielding the viewing window is repeated by putting the shield glass down. The above method is considerably complicated and increases fatigue of arms.

Further, according to the related art, since the welding helmet is supported while being worn in a head part of a worker, if the worker bends or bridles, a weight of the welding helmet concentrates upon a neck part of the worker so that fatigue of the worker is significantly increased during the welding operation.

### SUMMARY OF THE INVENTION

The present disclosure has been made in view of the above problems, and provides a welding helmet including a wireless auto light shield part capable of improving convenience of a welding operation, protecting eyes of a worker from direct light during welding operation, and preventing frost from being formed on the viewing window by shielding or opening a viewing window through elevation of a shield glass while automatically driving the light shield part in a wireless scheme by operation of a torch switch, and a method of controlling the same.

The present invention further provides a welding helmet including a wireless auto light shield part for preventing frost from being formed on the viewing window so that air may circulate inside the welding helmet, and a method of controlling the same.

The above information disclosed in this background section is only for enhancement of understanding of the background of the invention and therefore it may contain information that does not form the prior art that is already known in this country to a person of ordinary skill in the art.

### Technical Solution

In accordance with an aspect of the present invention, there is provided a welding helmet including a wireless auto light shield part connected to a welder body through a cable, and is used together with a torch including a torch switch during welding, the welding helmet including: a helmet body including a rear surface which is open, a space portion being formed at an inner side the helmet body, and a viewing window is provided at a front surface of the helmet body; a light shield glass located rearward of the viewing window; a light shield part configured to elevatably connect the light shield glass to the helmet body, and to open or shield the viewing window by elevating the light shield glass; a driving part configured to transfer power to the light shield part; a first wireless communication unit configured to receive a switching signal according to an on/off operation of the torch switch; and a first control unit configured to receive the switching signal from the first wireless communication unit to control the driving part so that the viewing window is open or shielded while elevating the light shield glass.

The welding helmet including a wireless auto light shield part may further include: a second wireless communication unit configured to detect an on/off operation of the torch switch; and a second control unit configured to receive the switching signal from the second wireless communication unit to control drive of the welder body so that welding starts or is terminated.

The welding helmet including a wireless auto light shield part may further include: a lighting part provided in the helmet body to illuminate a front direction of the helmet body, wherein the lighting part is located at an upper portion of the viewing window and includes: an LED module including a plurality of LEDs provided at a front surface of the helmet body and a printed circuit board connected with the LEDs; a power supply configured to supply power to the LED module; and a power switch configured to turn-on/off the LEDs.

The welding helmet including a wireless auto light shield part may further include: a wearing part worn on a shoulder of a worker to support the helmet body, wherein the wearing part includes a pair of wearing bars having a curved shape where a center thereof protrudes upward, a wearing frame including a connection bar configured to connect rear ends of the pair of wearing bars to each other, a connector configured to connect the wearing frame to the helmet body, and a wearing portion configured to wear the wearing frame in the worker.

The connector may include: a pair of first connection members provided at the pair of wearing bars, respectively; a pair of second connection members provided at both sides of the helmet body, respectively; and a pair of flexible bars including bottom ends connected to the first connection member and top ends connected to the second connection member, respectively, wherein each of the pair of first connection members may include: a first frame including both ends and one end connected to the wearing bar, a second frame including both ends and an opposite end connected to a bottom end of the flexible bar, and a first control lever configured to connect an opposite end of the first frame to one end of the second frame, and to control rotation of the second frame, and each of the pair of second connection members includes a third frame including both ends and one end connected to a top end of the flexible bar, and a second control lever configured to connect an opposite end of the third frame to the helmet body, and to control rotation of the helmet body.

The driving part may include a drive motor, and the light shield part includes first and second guide bars which are located both sides of a rear surface of the viewing window and including top and bottom ends fixed to upper and lower portions of the inner casing, a guide frame configured to elevatably connect an opposite side of the light shield glass to the second guide bar, and a drive assembly configured to elevatably connect one side of the light shield glass with the first guide bar, to connect the driving part with the light shield glass, and to elevate the light shield glass according to drive of the driving part.

The drive assembly may include: a rack configured to elevatably connect one side of the light shield glass to the first guide bar; and a pinion configured to receive a rotating force from the driving part to elevate the rack.

The light shield part may further include upper and lower limit sensors making contact with the light shield glass to stop drive of the drive motor when the light shield glass elevates to a lower limit position or an upper limit position.

The welding helmet including a wireless auto light shield part may further include a blowing fan for preventing frost to be formed on the viewing window by exhausting internal air of the helmet body to an exterior.

In accordance with another aspect of the present invention, there is provided a method of controlling a welding helmet including a wireless auto light shield part, the method including: receiving an ON signal of a torch switch to transfer the ON signal to the first and second control units, respectively by the first wireless communication unit and the second wireless communication unit when the torch switch is turned-on; the light shield glass falls a lower limit position according to drive of the driving part to shield the viewing window when the ON signal of the torch switch is input to the first control unit; the welder body is operated to supply power to the torch so that welding starts when the ON signal of the torch switch is input to the second control unit; receiving an OFF signal of the torch switch to transfer the OFF signal to the first and second control units, respectively by the first wireless communication unit and the second wireless communication unit when the torch switch is turned-off; driving the driving part to lift the light shield glass to an upper limit position to open the viewing window when the OFF signal of the torch switch is input to the first control unit; and stopping the drive of the welder body to block supply of power to the torch so that the welding is not performed when the OFF signal of the torch switch is input to the second control unit.

The second control unit may drive the welder body when a predetermined time elapses when the ON signal of the torch switch is received.

The first control unit may drive the driving part when a predetermined time elapses after the OFF signal of the torch switch is received.

### Effects of the Present Invention

The present invention can improve convenience of a welding operation, protect eyes of a worker from direct light upon welding, and prevent frost from being formed on the viewing window by shielding or opening a viewing window through elevation of a shield glass while automatically driving a shield part by operation of a torch switch without including a separate operation switch.

Further frost can be prevented from being formed on the viewing window so that air may circulate inside the welding helmet.

Meanwhile, other various effects may be directly or indirectly disclosed in the following description of the embodiment of the disclosure.

### BRIEF DESCRIPTION OF THE DRAWINGS

FIG. 1 is a conceptual diagram schematically illustrating a welding helmet including a wireless auto light shield part according to the present invention.

FIG. 2 is a perspective view illustrating a helmet body of the welding helmet including a wireless auto light shield part according to the present invention.

FIG. 3 is a perspective view illustrating a wearing part of the welding helmet including a wireless auto light shield part according to the present invention.

FIG. 4 is a rear view illustrating a helmet body of the welding helmet including a wireless auto light shield part according to the present invention.

FIG. 5 is an exploded perspective view illustrating a casing of the welding helmet including a wireless auto light shield part according to the present invention.

FIG. 6 is a rear view illustrating a light shield part of the welding helmet including a wireless auto light shield part according to the present invention.

FIG. 7 is a flowchart illustrating a method of controlling a welding helmet including a wireless auto light shield part according to the present invention.

### DESCRIPTION OF EXEMPLARY EMBODIMENTS

Hereinafter, preferred embodiments of the present invention will be described with reference to accompanying drawings.

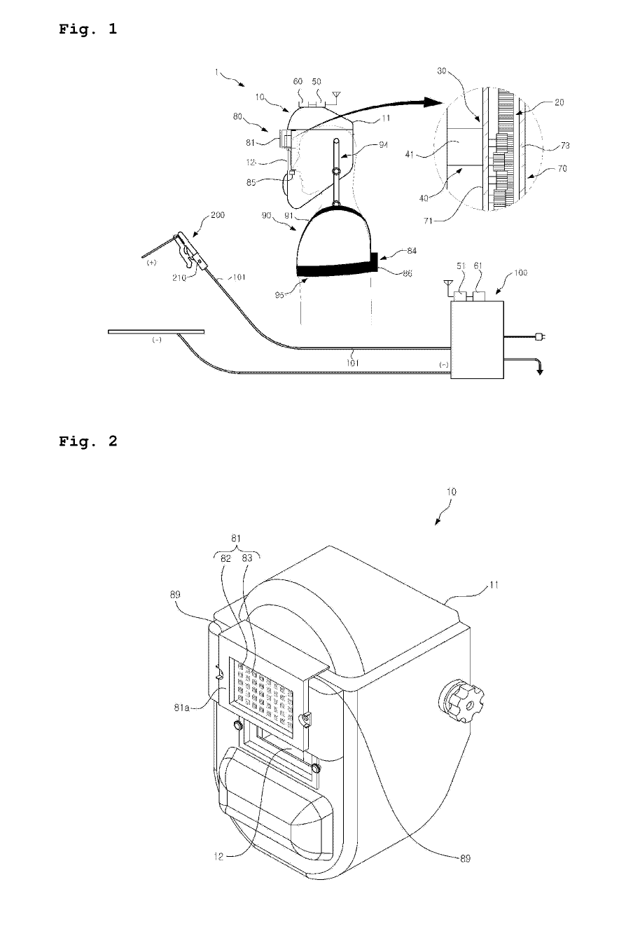

FIG. 1 is a conceptual diagram schematically illustrating a welding helmet including a wireless auto light shield part according to the present invention. Referring to FIG. 1, a welding helmet 1 including a wireless auto light shield part (hereinafter referred to as ‘welding helmet’) is connected to a welder body 100 through a cable 101, and is used together with a torch 200 including a torch switch 210 during welding, and includes: a helmet body 10, a light shield glass 20, a light shield part 30, a driving part 40, a first wireless communication unit 50, and a first control unit 60.

A rear surface of the helmet body 10 is open, a space portion 11 is formed at an inner side the helmet body 10, and a viewing window 12 is provided at a front surface of the helmet body 10. The light shield glass 20 is located rearward of the viewing window 12. The light shield part 30 elevatably connects the light shield glass 20 to the helmet body 10, and opens or shields the viewing window 12 by elevating the light shield glass 20.

The driving part 40 transfers power to the light shield part 30. The first wireless communication unit 50 receives a switching signal according to an on/off operation of the torch switch 210. The first control unit 60 receives the switching signal from the first wireless communication unit 50 to control the driving part 40 so that the viewing window 12 is open or shielded while elevating the light shield glass 20.

A head of a worker is received in the space portion 11. The worker ensures a sight through the viewing window 12 while a face makes contact with or is close to an inner surface forming the space portion 11. The welder body 100 receives power from an exterior to generate a voltage for generating each electrode. The torch 200 is connected to the welding body through the cable 101, and an electrode is installed at the torch 200. If the torch switch 210 is turned-off in a state that the power from the welder body 100 is applied to the torch switch 210, the light shield part 30 lifts the light shield glass 20 under drive of the driving part 40 to open the viewing window 12. Further, if the torch switch 210 is turned-on, the light shield part 30 falls the light shield glass 20 under drive of the driving part 40 to shield the viewing window 12.

The welder body 100 receives power from an exterior to generate a voltage for generating each electrode. The welder body 100 receives a switching signal according to an on/off operation of the torch switch 210 to be turned-on/off. To this end, the welding helmet includes a second wireless communication unit 51 configured to detect an on/off operation of the torch switch 210 and a second control unit 61 configured to receive the switching signal from the second wireless communication unit 51 to control drive of the welder body so that welding starts or is terminated. It is preferable that the second wireless communication unit 51 and the second control unit 61 are included in the welder body, and may receive power from the welder body 100.

FIG. 2 is a perspective view illustrating a helmet body of the welding helmet including a wireless auto light shield part according to the present invention. Referring to FIG. 2, the welding helmet 1 further includes a lighting part 80 which is provided in the helmet body 10. The lighting part 80 is located at an upper portion of the viewing window 12 and includes an LED module 81, a power supply 84, and a power switch 85. The LED module 81 includes a plurality of LEDs 82 provided at a front surface of the helmet body 10 and a printed circuit board (PCB) 83 connected with the LEDs 82. The power supply 84 supplies power to the LED module 81. The power switch 85 turns-on/off the LEDs 82. The power supply 84 includes a battery 86 and is connected to the power switch 85 and the LED module 81 through a power wire (not shown). The power supply 84 and the power switch 85 may be included in the helmet body 110 or a wearing part 90 to be described later. It is preferable that the LED module 81 is disposed forward of the viewing window 12. The LED module 81 is disposed forward of the viewing window 12 for the purpose of preventing glare due to the LEDs 82. The lighting part 80 may include a cover 81a configured to cover the LED module 81. The cover 81a may protect the LED module 81 and may allow light illuminated from the LEDs 81 to widely spread.

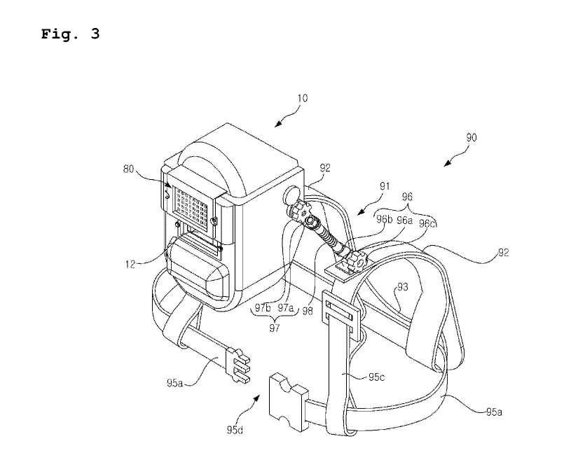

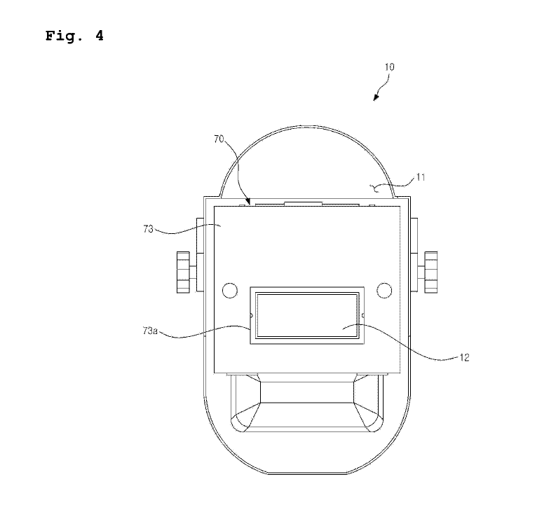

FIG. 3 is a perspective view illustrating a wearing part of the welding helmet including a wireless auto light shield part according to the present invention. Referring to FIG. 3, the welding helmet further includes a wearing part 90 worn on a shoulder of a worker to support the helmet body 10. The wearing part 90 supports the helmet body 10 not to transfer a weight of the helmet body 10 to a head of the worker. To this end, the wearing part 90 includes a pair of wearing bars 92 having a curved shape where a center thereof protrudes upward, a wearing frame 91 including a connection bar 93 configured to connect rear ends of the pair of wearing bars 91 to each other, a connector 94 configured to connect the wearing frame 91 to the helmet body 10, and a wearing part 95 configured to wear the wearing frame 91 in a worker. The pair of wearing bars 92 is placed on both shoulders of the worker.

The connection bar 93 is located at a rear side of a body of the worker. The connector 94 includes a pair of first connection members 96 provided at the pair of wearing bars 92, respectively, a pair of second connection members 97 provided at both sides of the helmet body 10, respectively, and a pair of flexible bars 98 including bottom ends connected to the first connection member 96 and top ends connected to the second connection member 97, respectively. The pair of the flexible bars 98 support the helmet body 10 from the wearing frame 91 and are bent due to external pressing. Each of the pair of first connection members 96 include a first frame 96 including both ends and one end connected to the wearing bar 91, a second frame 96b including both ends and an opposite end connected to a bottom end of the flexible bar 98, and a first control lever 96c configured to connect an opposite end of the first frame 96a to one end of the second frame 96b, and to control rotation of the second frame 96b. The first control lever 96c is screwed to the first and second frames 96a and 96b. According to tightening and looseness, the first control lever 96c controls rotation of the helmet body 10 synchronized to rotation of the second frame 96b.

Each of the pair of second connection members 97 includes a third frame 97a including both ends and one end connected to a top end of the flexible bar 98, and a second control lever 97b configured to connect an opposite end of the third frame 97a to the helmet body 10, and to control rotation of the helmet body 10. The second control lever 97b is screwed to the third frame 97a and controls rotation of the helmet body 10 according to tightening and looseness. The wearing part 95 includes a body string 95a connected to a front surface of the connection bar 93, a pair of shoulder strings 95b including a rear end connected to the body string 95a and a front end extending along the wearing bar 92, a pair of cohesion strings 95c configured to connect the pair of shoulder strings 95b to both ends of the body string 95a, and a cohesion member 95d configured to cohere the both ends of the body string 95a to each other or to release the cohesion. The cohesion member 95d may include male and female buckles.

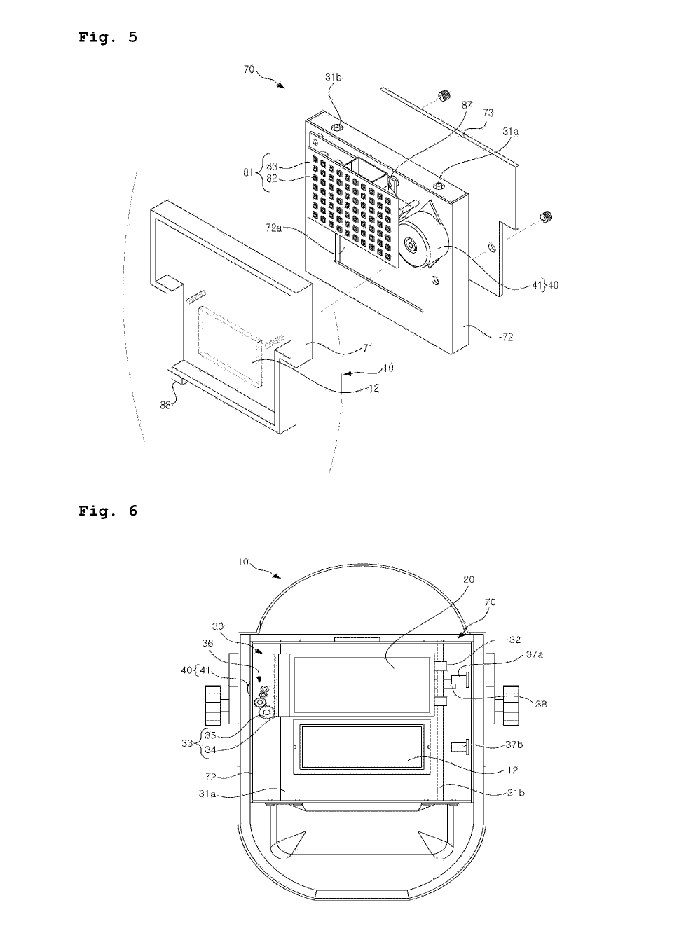

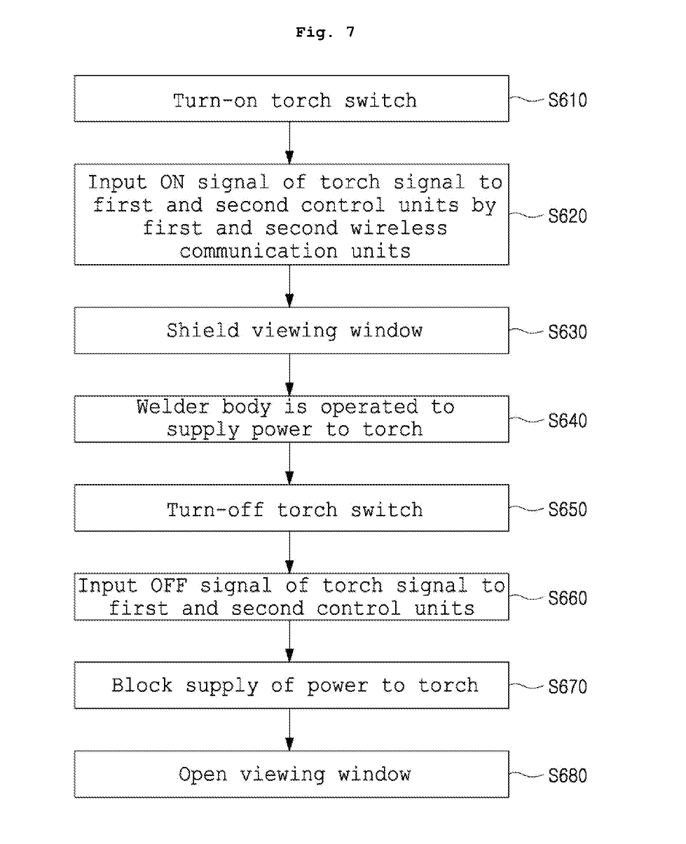

FIG. 4 is a rear view illustrating a helmet body of the welding helmet including a wireless auto light shield part according to the present invention. FIG. 5 is an exploded perspective view illustrating a casing of the welding helmet including a wireless auto light shield part according to the present invention, and FIG. 6 is a rear view illustrating a light shield part of the welding helmet including a wireless auto light shield part according to the present invention.

Referring to FIG. 4 to FIG. 6, the welding helmet further includes a casing 70 configured to receive the light shield part 30 at an inner surface of the space portion 11, that is, a rear surface of the helmet body 10.

The casing 70 is provided at a rear surface of the helmet body 10. The casing 70 includes a lateral frame 71 located at an outer side of the viewing window 12, an inner casing 72 coupled with an inner side of the lateral frame 71 and formed therein with a first hollow region 72a communicating with the viewing window 12, including an open rear surface, and fixed to the light shield part 30 and the driving part 40, and a cover 73 configured to cover the open rear surface of the inner casing 72 and formed therein with a second hollow region 73a communicating with the first hollow 72a.

The light shield part 30 is provided at an inner side of the casing 70 so that the light shield glass 20 elevates rearward of the viewing window 21 according to drive of the driving part 40.

The light shield part 30 includes first and second guide bars 31a and 31b which are located both sides of a rear surface of the viewing window 21 and including top and bottom ends fixed to upper and lower portions of the inner casing 72, a guide frame 32 configured to elevatably connect an opposite side of the light shield glass 20 to the second guide bar 31b, and a drive assembly 33 configured to elevatably connect one side of the light shield glass 20 with the first guide bar 31a, to connect the driving part 40 with the light shield glass 20, and to elevate the light shield glass 20 according to drive of the driving part 40.

The driving part 40 includes a drive motor 41, and is fixed to a front surface of the inner casing 72 and receives power from the battery 86.

The drive assembly 33 includes a rack 34 configured to elevatably connect one side of the light shield glass 20 to the first guide bar 31a and a pinion 35 configured to receive a rotating force from the driving part 40 to elevate the rack 34.

The light shield part 30 further includes a reducer 36 configured to connect the pinion 35 to the drive motor 41. The reducer 36 may include a plurality of gears which are tooth-bonded to each other.

The drive motor 41 transfers a rotating force in forward and reverse directions. When the drive motor 41 is operated, the pinion 35 is rotated so that the rack 34 elevates in connection with the rack 34.

When the rack 34 elevates, the light shield glass 20 elevates together with the guide frame 32 connected to the rack 34 to open or shield the viewing window 12.

The welding helmet may include a blowing fan 87 for preventing frost to be formed on the viewing window 12.

The blowing fan 87 may prevent frost from being formed on the viewing window 12 by easily circulate air inside the helmet body 10. As shown in FIG. 5, it is preferable that the blowing fan 87 is provided at a front surface of the inner casing 72 and receives power from the battery 86 to be operated.

Further, a switch 88 for turning-on/off the blowing fan 87 may be provided at the lateral frame 71.

As shown in FIG. 2, the helmet body 10 is formed therein with an air outlet 89 to exhaust air introduced from the blowing fan 87 to the cover 81a.

Accordingly, the blowing fan 87 is operated to introduce internal air of the helmet body 10 and exhaust the introduced internal air through the air outlet 89, thereby preventing frost to be formed on the viewing window 12.

It is preferable that the first wireless communication unit 50 and the first control unit 60 are provided at the helmet body 10. The first wireless communication unit 50 receives a switching signal according to an on/off operation of the torch switch 21 to provide the switching signal to the first control unit 60.

The first control unit 60 receives the switching signal from the first wireless communication unit 50 to control the driving part 40 to elevate the light shield glass 20, thereby opening or shielding the viewing window 12.

The light shield part 30 further includes upper and lower limit sensors 37a and 37b configured to detect the signal according to opening or shielding of the viewing window 12 so that the first control unit 60 stops drive of the drive motor 41. The light shield part 30 further includes a guide piece 38 elevating together with the light shield glass 20, and making contact with the upper and lower limit sensors 37a and 37b so that the upper and lower limit sensors 37a and 37b detect an opening or shielding state of the viewing window 12.

The upper and lower limit sensors 37a and 37b are located at upper and lower portions of an opposite side of the light shield glass 20. The guide piece 38 protrudes outward of the opposite side of the light shield glass 30. When the light shield glass 30 elevates to a lower limit position or an upper limit position, the guide piece 38 makes contact with the upper limit sensor 37a or the lower limit sensor 37b so that the upper and lower limit sensors 37a and 37b detect the opening or shielding state of the viewing window 21. In other words, the upper and lower limit sensors 37a and 37b detect whether the light shield glass 30 reaches the lower limit position or the upper limit position.

The first control unit 60 receives a switching signal from the first wireless communication unit 50 according to on/off of the torch switch 210 to drive the drive motor 41, and blocks power to be supplied to the drive motor 41 from the battery 86 according to a detection signal of the upper and lower limit sensors 37a and 37b to stop drive of the drive motor 41.

Accordingly, the drive motor 41 stops under control of the first control unit 60 in a state that the light shield glass 20 elevates to the lower limit position or the upper limit position so that the viewing window 12 may maintain the opening or shielding state.

FIG. 7 is a flowchart illustrating a method of controlling a welding helmet including a wireless auto light shield part according to the present invention. Referring to FIG. 7, first, after a worker wears the helmet body 10 adhering to a face of the worker, the torch switch 210 is turned-on (S610). When the torch switch 210 is turned-on, the first wireless communication unit 50 included in the helmet body 10 and the second wireless communication unit 51 included in the welder body 100 receive an ON signal of the torch switch 210 to transfer the ON signal to the first and second control units 60 and 61, respectively (S620). If the ON signal of the torch switch 210 is input to the first control unit 60, the light shield glass 20 falls a lower limit position according to drive of the driving part 40 to shield the viewing window 12 (S630). Next, the first control unit 60 stops drive of the driving part 40 according to a detection signal of the lower limit sensor 37b. Further, if the ON signal of the torch switch 210 is input to the second control unit 61, the welder body 100 is operated to supply power to a torch 200 so that welding may start (S640).

In this case, the second control unit 61 drives the welder body 100 when a predetermined time elapses when the ON signal of the torch switch 210 is received. If the torch switch 210 is pushed to be in an on state, the driving part 40 is operated to shield the viewing window 12 by falling the light shield glass 20 so that a control signal is output to the welder body 100 to start the welding in a state that the worker' eye may be protected from direct light omitted upon the welding.

Next, when the torch switch 210 is turned-off (S650), the first wireless communication unit 50 included in the helmet body 10 and the second wireless communication unit 51 included in the welder body 100 receive an Off signal of the torch switch 210 to transfer the Off signal to the first and second control units 60 and 61, respectively (S660). If the OFF signal of the torch switch 210 is input to the second control unit 61, the drive of the welder body 100 stops to block supply of power to the torch 20 so that the welding may not be performed (S670). If the OFF signal of the torch switch 210 is input to the first control unit 60, the driving part 40 is operated after a predetermined time elapses so that the light shield glass 20 lifts to the upper limit position to open the viewing window 12 (S680). Further, the first control unit 60 stops the drive of the driving part 40 according to a detection signal of the upper limit sensor 37a.

In this case, the first control unit 60 drives the driving part 40 when a predetermined time elapses after the OFF signal of the torch switch 210 is received. If the torch switch 210 is turned-off, the drive of the welder body 100 stops to block supply of power to the torch 200 and the light shield glass 20 lifts to open the viewing window 12 so that the worker's eyes may be protected.

Although the welding helmet including a wireless auto light shield part and a controlling method thereof according to embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

* Description of reference numerals*

| 1: Welding helmet | 10: Helmet body |
|---|---|
| 11: Space portion | 12: Viewing window |
| 20: Light shield glass | 30: Light shield part |
| 31a: First guide bar | 31b: Second guide bar |
| 32: Guide frame | 33: Drive assembly |
| 34: Rack | 35: Pinion |
| 36: Reducer | 37a: Upper limit sensor |
| 37b: Lower limit sensor | 38: Guide piece |
| 31: Guide bar | 40: Driving part |
| 50: First wireless communication unit | |
| 51: Second wireless communication unit | |
| 60: First control unit | 61: Second control unit |
| 70: Casing | 80: Lighting part |
| 81: LED module | 84: Power supply |
| 86: Battery | 87: Blowing fan |
| 90: Wearing portion | 91: Wearing frame |
| 92: Wearing bar | 93: Connection bar |
| 94: Connector | 95: Wearing part |
| 96: First connection member | |
| 97: Second connection member | |
| 98: Flexible bar | 100: Welder body |
| 200: Torch | 210: Torch switch |

1. A welding helmet including a wireless auto light shield part connected to a welder body through a cable, and is used together with a torch including a torch switch during welding, the welding helmet comprising:
a helmet body including a rear surface which is open, a space portion being formed at an inner side the helmet body, and a viewing window is provided at a front surface of the helmet body;
a light shield glass located rearward of the viewing window;
a light shield part configured to elevatably connect the light shield glass to the helmet body, and to open or shield the viewing window by elevating the light shield glass;
a driving part configured to transfer power to the light shield part;
a first wireless communication unit configured to receive a switching signal according to an on/off operation of the torch switch; and
a first control unit configured to receive the switching signal from the first wireless communication unit to control the driving part so that the viewing window is open or shielded while elevating the light shield glass.

2. The welding helmet including a wireless auto light shield part of claim 1, further comprising:
a second wireless communication unit configured to detect an on/off operation of the torch switch; and
a second control unit configured to receive the switching signal from the second wireless communication unit to control drive of the welder body so that welding starts or is terminated.

3. The welding helmet including a wireless auto light shield part of claim 2, further comprising:
a lighting part provided in the helmet body to illuminate a front direction of the helmet body,
wherein the lighting part is located at an upper portion of the viewing window and includes:
an LED module including a plurality of LEDs provided at a front surface of the helmet body and a printed circuit board connected with the LEDs;
a power supply configured to supply power to the LED module; and
a power switch configured to turn-on/off the LEDs.

4. The welding helmet including a wireless auto light shield part of claim 2, further comprising: a wearing part worn on a shoulder of a worker to support the helmet body,
wherein the wearing part includes a pair of wearing bars having a curved shape where a center thereof protrudes upward, a wearing frame including a connection bar configured to connect rear ends of the pair of wearing bars to each other, a connector configured to connect the wearing frame to the helmet body, and a wearing portion configured to wear the wearing frame in the worker.

5. The welding helmet including a wireless auto light shield part of claim 4, wherein the connector comprises:
a pair of first connection members provided at the pair of wearing bars, respectively;
a pair of second connection members provided at both sides of the helmet body, respectively; and
a pair of flexible bars including bottom ends connected to the first connection member and top ends connected to the second connection member, respectively,
wherein each of the pair of first connection members includes:
a first frame including both ends and one end connected to the wearing bar,
a second frame including both ends and an opposite end connected to a bottom end of the flexible bar, and
a first control lever configured to connect an opposite end of the first frame to one end of the second frame, and to control rotation of the second frame, and
each of the pair of second connection members includes a third frame including both ends and one end connected to a top end of the flexible bar, and
a second control lever configured to connect an opposite end of the third frame to the helmet body, and to control rotation of the helmet body.

6. The welding helmet including a wireless auto light shield part of claim 2, wherein the driving part includes a drive motor, and
the light shield part includes first and second guide bars which are located both sides of a rear surface of the viewing window and including top and bottom ends fixed to upper and lower portions of the inner casing,
a guide frame configured to elevatably connect an opposite side of the light shield glass to the second guide bar, and a drive assembly configured to elevatably connect one side of the light shield glass with the first guide bar, to connect the driving part with the light shield glass, and to elevate the light shield glass according to drive of the driving part.

7. The welding helmet including a wireless auto light shield part of claim 6, wherein the drive assembly includes:
a rack configured to elevatably connect one side of the light shield glass to the first guide bar; and
a pinion configured to receive a rotating force from the driving part to elevate the rack.

8. The welding helmet including a wireless auto light shield part of claim 6, wherein the light shield part further includes upper and lower limit sensors making contact with the light shield glass to stop drive of the drive motor when the light shield glass elevates to a lower limit position or an upper limit position.

9. The welding helmet including a wireless auto light shield part of claim 2, further comprising a blowing fan for preventing frost to be formed on the viewing window by exhausting internal air of the helmet body to an exterior.

10. A method of controlling a welding helmet including a wireless auto light shield part according to claim 2, the method comprising:
receiving an ON signal of a torch switch to transfer the ON signal to the first and second control units, respectively by the first wireless communication unit and the second wireless communication unit when the torch switch is turned-on;
the light shield glass falls a lower limit position according to drive of the driving part to shield the viewing window when the ON signal of the torch switch is input to the first control unit;
the welder body is operated to supply power to the torch so that welding starts when the ON signal of the torch switch is input to the second control unit;
receiving an OFF signal of the torch switch to transfer the OFF signal to the first and second control units, respectively by the first wireless communication unit and the second wireless communication unit when the torch switch is turned-off;
driving the driving part to lift the light shield glass to an upper limit position to open the viewing window when the OFF signal of the torch switch is input to the first control unit; and
stopping the drive of the welder body to block supply of power to the torch so that the welding is not performed when the OFF signal of the torch switch is input to the second control unit.

11. The method of claim 10, wherein the second control unit drives the welder body when a predetermined time elapses when the ON signal of the torch switch is received.

12. The method of claim 10, wherein the first control unit drives the driving part when a predetermined time elapses after the OFF signal of the torch switch is received.
